(19) United States
(12) Patent Application Publication
(10) Pub. No.: US 2020/0120910 A1
(43) Pub. Date: Apr. 23, 2020

(54) TRIPLOID SHELLFISH

(71) Applicant: Green Mountain Aquaculture, Inc., San Diego, CA (US)

(72) Inventors: Harrison MACDONALD, San Diego, CA (US); Nicolas LAQUA, New York, NY (US)

(21) Appl. No.: 16/657,202

(22) Filed: Oct. 18, 2019

Related U.S. Application Data
(60) Provisional application No. 62/807,371, filed on Feb. 19, 2019.
(60) Provisional application No. 62/772,992, filed on Nov. 29, 2018.
(60) Provisional application No. 62/748,240, filed on Oct. 19, 2018.

(51) Int. Cl. A01K 67/033; A01K 61/54

(52) U.S. Cl. CPC A01K 67/0334; A01K 61/54

(57) ABSTRACT

The present disclosure describes systems and methods for producing triploid shellfish.

### CROSS REFERENCE

This application claims the benefit of U.S. Provisional Application No. 62/807,371 filed on Feb. 19, 2019, U.S. Provisional Application No. 62/772,992 filed on Nov. 29, 2018, and U.S. Provisional Application No. 62/748,240 filed on Oct. 19, 2018, the contents of each of which is incorporated herein by reference in its entirety.

### BACKGROUND

Rising shellfish consumption has increased consumer demand and driven costs upward. Improved shellfish farming methods are needed to increase efficiency, address economical costs, and meet the increasing demand.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

The present invention relates to producing triploid shellfish organisms by cross mating tetraploid and diploid shellfish organisms.

Commercial shellfish are filter feeder organisms that largely consume microalgae, such as phytoplankton and zooplankton. Shellfish are exoskeleton-bearing aquatic invertebrates that include shelled mollusks, crustaceans, and echinoderms. Non-limiting examples of mollusks include oysters, clams, mussels, cockles, scallops, abalone, and winkles.

Each individual shellfish filters more than fifty gallons of seawater per day. Thus, shellfish deter algal blooms that pose ecological problems in marine habitats.

Shellfish sexually reproduce and exist in the wild as diploids. Through biotechnological approaches, triploid shellfish having three chromosomes, one from the male and two from the female or vice versa, can be produced. Triploid shellfish are sexually sterile and do not spawn, i.e. produce eggs or sperm. Thus, triploid shellfish are available for harvest year-round as meat yield is more consistent than wild type, diploid shellfish. In contrast, diploid shellfish grow seasonally. Diploids typically have low summertime meat yield as a result of spawning activities in the wintertime. Shellfish can devote over half their body weight to the production of gametes during spawning. For this reason, triploid shellfish can grow about 50% faster than the diploid counterparts. As a result, triploid shellfish meat is plumper, less watery, and tastier than that of wild type equivalents.

As shellfish sustain by filtering large quantities of seawater, shellfish can accumulate microorganisms and pollutants that promote susceptibility to disease and high mortality rates. For example, oyster-borne diseases affect the gonads. Due to lack of reproductive organs, triploid shellfish are more resistant to disease than the wild diploid counterparts. The faster rate of growth of triploids also contributes to disease resistance by limiting the time span in which the organism is exposed to pathogens.

The Philippines hooded oyster Saccostrea palmipes is a benthic bivalve mollusk that is native to Western Pacific Japan and the Philippines. Saccostrea palmipes spawns routinely and is diecious. Fertilization of Saccostrea palmipes occurs externally, with no secondary sexual characteristics apart from gonadal tissue. Fertilized eggs of Saccostrea palmipes are approximately 40 μm in diameter. Within 5 hours of fertilization, the eggs hatch into free swimming larvae known as trochophores. Within 35 hours of fertilization, the trochophores form two shells. The larvae freely swim for approximately 2 to 3 weeks, during which they are dispersed by ocean currents. Once the larvae reach about 2.5 mm in size, the larvae metamorphose and cement themselves to a permanent surface. The larvae are sexually mature within 4 months.

The wild type, diploid Philippines hooded oyster has 20 chromosomes. Mature eggs are arrested at the prophase of the first meiotic division. At this stage, 10 synapsis tetrads can be visualized in the eggs when examined under a microscope. After activation, the 10 tetrads undergo two meiotic divisions and release two polar bodies: polar body I containing 20 dyads and polar body II containing 10 chromatids. The remaining 10 chromatids in the oocyte unite with the 10 chromatids from the sperm to form a diploid zygote.

Tetraploid oysters can be produced from a triploid female and a diploid male. Chromosome set manipulation can facilitate this process in which triploid females can be conditioned by placing them in an environment with diploid males. Commercially-available triploid Pacific oysters are typically produced by breeding tetraploid Pacific oysters with diploid Pacific oysters.

### Triploid Production

Triploid shellfish can be produced by breeding tetraploids with diploids.

Tetraploids production can be induced by treatment with cytochalasin B (CB), which inhibits triploid embryo formation so that haploid sperm can fertilize triploid eggs to produce tetraploid embryos. In some embodiments, tetraploids are treated with about 0.5 mg/L, about 0.6 mg/L, about 0.7 mg/L, about 0.8 mg/L, about 0.9 mg/L, or about 10 mg/L of CB.

Spawning can then be induced by thermal shock, which causes haploid sperm from wild type, diploids to fertilize tetraploid eggs.

The fertilized larvae can be cultured in water sterilized by UV radiation and 1-μm filtration, and supplemented with flagellates and diatoms. Metamorphosis can be induced by application of norepinephrine and partial shell powder, followed by upwelling and downwelling-facilitated growth.

In some embodiments, a triploid shellfish species produced by methods disclosed herein is about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300% larger in size than the corresponding wild type diploid shellfish species. In some embodiments, a triploid shellfish species produced by methods disclosed herein is more than 300% larger in size than the corresponding wild type diploid shellfish species.

In some embodiments, the growth rate of a triploid shellfish species produced by methods disclosed herein is about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, about 300%, about 400%, about 500%, about 600%, about 700%, about 800%, about 900%, about 1000%, about 1100%, about 1200%, about 1300%, about 1400%, about 1500%, about 1600%, about 1700%, about 1800%, about 1900%, about 2000%, about 3000% about 2000%, about 3000%, about 4000%, or about 5000% faster than the growth rate of the corresponding wild type diploid shellfish species. In some embodiments, the growth rate of a triploid shellfish species produced by methods disclosed herein is more than 5000% faster than the growth rate of the corresponding wild type diploid shellfish species.

### Shellfish Species

Non-limiting examples of shellfish genus and species are listed in TABLE 1.

TABLE 1

| Genus | Species | Common name |
|---|---|---|
| Ostrea | Ostrea edulis | European flat oyster |
| | Ostrea iridescens | |
| | Ostrea palmula | |
| | Ostrea folium | |
| Crassostrea | Crassostrea rhizophorae | Mangrove oyster |
| | Crassostrea brasiliana | Mangrove oyster |
| | Crassostrea ariakensis | Suminoe oyster |
| Saccostrea | Saccostrea malabonensis | Philippines hood oysters |
| | Saccostrea palmipes | |
| | Saccostrea echinata | |
| Anadara | Anadara granosa | Blood cockle (clam) |
| Venerupis | Venerupis philippinarum | Manila clam |
| Mercenaria | Mercenaria mercenaria | Quahog clam |
| Dreissena | Dreissena polymorpha | Zebra mussel |
| | Dreissena bugensis | Quagga mussel |
| Mytilus | Mytilus edulis | Blue mussel |
| | Mytilus galloprovincialis | Mediterranean mussel |
| | Mytilus coruscus | Korean mussel |
| | Mytilus chilensis | Chilean mussel |
| Perna | Perna viridis | Asian green mussel |
| | Perna canaliculus | New Zealand mussel |
| Argopecten | Argopecten irradians | Bay scallop |
| | Argopecten purpuratus | Peruvian scallop |
| Aequipecten | Aequipecten opercularis | Queen scallop |
| Patinopecten | Patinopecten yessoensis | Japanese scallop |

### EXAMPLES

### Example 1

### Production of Tetraploid Oysters

Triploid eggs can be fertilized with haploid sperm derived from diploids. Triploid females can be conditioned by placing in an environment with high temperature and abundant food. Triploid oysters can be produced from wild type, diploid oysters by inhibiting the release of polar body II from fertilized eggs during meiosis. The ploidy of the oysters can be confirmed by flow cytometry prior to spawning. Gametes can be obtained by strip-spawning. The eggs can then be fertilized with sperm obtained from a normal diploid male. Gamete ratio can be adjusted for optimal fertilization rates. For example, the sperm to egg fertilization ratio can be at least about 10:1 to about 100:1.

After fertilization, the release of polar body I from the eggs can be performed by treating the eggs with a blocking agent, such as cytochalasin B (CB). Alternative blocking methods, for example, include 6-dimethylaminopurine (6-DMAP) treatment, thermal shocking, or hydrostatic shocking of the fertilized eggs. Depending on the shellfish species and temperature, CB treatment duration and dosage can be adjusted to optimize results. Typically, the duration of CB treatment is about 60-80% of the average time required for half of the untreated triploid eggs to release polar body I in the absence of CB under the same conditions. For example, CB treatment for Pacific oysters can last about 15-20 min at 25° C.

Following CB treatment, the eggs can be rinsed to remove excess CB and subjected to standard shellfish growth processes to produce tetraploid oysters.

### Example 2

### Production of Triploid Philippines Hood Oysters

Tetraploids and wild type, diploid Philippines hood oysters were crossmated to produce triploid Philippines hood oysters. Similar to diploids, tetraploids mature at one year of age. Diploid eggs were mated with tetraploid sperm cells. Conversely, tetraploid eggs can be mated with haploid sperm from wild type, diploids. The gamete ratio can be adjusted to optimize fertilization rates. For example, the sperm to egg fertilization ratio can be from about 10:1 to about 100:1.

One-year old triploid Philippines hooded oysters Saccostrea malabonensis were produced by crossmating tetraploids with diploids by blocking PB2. Oyster ploidy was confirmed via flow cytometry. After confirming triploid identity, the gametes were isolated from the oysters. The eggs were rinsed on a 25-μm screen. Fertilization and treatment processes were conducted at 29° C. in seawater having a salinity of 23 ppt. The isolated triploid eggs were fertilized with haploid sperm from wild type, diploids. After fertilization, the eggs were treated with CB for 15 min to block the release of PB1. CB was prepared in 0.5% dimethyl sulfoxide (DMSO) and added to fertilized eggs at a final concentration of 0.8 mg/L. After CB treatment, the eggs were rinsed with seawater to remove CB. The larvae were then cultured into spat within 25 days to adult size. Ploidy composition was determined via flow cytometry.

The oysters were grown to adult size. Average size and growth rates for wild type diploid and triploid Philippines hooded oysters are shown in TABLE 2. The triploids were about 59% larger than the corresponding diploids. The growth rate of triploids was about 1900% faster than the growth rate of the corresponding diploids.

### Example 3

### Production of Triploid Mediterranean Mussels

One-year old triploid Mediterranean mussel Mytilus galloprovincialis were produced by blocking PB2, were individually confirmed via flow cytometry. After the triploids are confirmed, gametes were obtained, and eggs were placed on a 25-μm Screen. Fertilization and treatment were conducted at 29° C. with seawater with a salinity of 23 ppt. Eggs from triploids were fertilized with haploid sperm from naturally occurring diploids. After fertilization, the eggs were exposed to TDCB where they were treated with cytochalasin B that was prepared in dimethyl sulfoxide and added at a concentration of 0.8 mg/L with 0.5% DMSO. This was done in order to block the release of PB1, and lasted for 15 minutes, at which point the eggs were rinsed with seawater and cultured. The larvae were cultured into spat within 25 days, at which point their composition was determined via flow cytometry.

The mussels were grown to adult size. Average size and growth rates for wild type diploid and triploid Mediterranean mussels are shown in TABLE 2. The triploids were about 55% larger than the corresponding diploids. The growth rate of triploids was about 900% faster than the growth rate of the corresponding diploids.

### Example 4

### Production of Triploid Quahog Clams

One-year old triploid Quahog clams Mercenaria mercenaria were produced by blocking PB2, were individually confirmed via flow cytometry. After the triploids are confirmed, gametes were obtained, and eggs were placed on a 25-μm Screen. Fertilization and treatment were conducted at 29° C. with seawater with a salinity of 23 ppt. Eggs from triploids were fertilized with haploid sperm from naturally occurring diploids. After fertilization, the eggs were exposed to TDCB where they were treated with cytochalasin B that was prepared in dimethyl sulfoxide and added at a concentration of 0.8 mg/L with 0.5% DMSO. This was done in order to block the release of PB1, and lasted for 15 minutes, at which point the eggs were rinsed with seawater and cultured. The larvae were cultured into spat within 25 days, at which point their composition was determined via flow cytometry.

The clams were grown to adult size. Average size and growth rates for wild type diploid and triploid Quahog clams are shown in TABLE 2. The triploids were about 48% larger than the corresponding diploids. The growth rate of triploids was about 800% faster than the growth rate of the corresponding diploids.

### Example 5

### Production of Triploid Queen Scallops

One-year old triploid Queen scallops Aequipecten opercularis were produced by blocking PB2, were individually confirmed via flow cytometry. After the triploids are confirmed, gametes were obtained, and eggs were placed on a 25-μm Screen. Fertilization and treatment were conducted at 29° C. with seawater with a salinity of 23 ppt. Eggs from triploids were fertilized with haploid sperm from naturally occurring diploids. After fertilization, the eggs were exposed to TDCB where they were treated with cytochalasin B that was prepared in dimethyl sulfoxide and added at a concentration of 0.8 mg/L with 0.5% DMSO. This was done in order to block the release of PB1, and lasted for 15 minutes, at which point the eggs were rinsed with seawater and cultured. The larvae were cultured into spat within 25 days, at which point their composition was determined via flow cytometry.

The scallops were grown to adult size. Average size and growth rates for wild type diploid and triploid Queen scallops are shown in TABLE 2. The triploids were about 58% larger than the corresponding diploids. The growth rate of triploids was about 900% faster than the growth rate of the corresponding diploids.

TABLE 2

| | Average Size (mm) | | | Average Growth Rate (mm/month) | | |
|---|---|---|---|---|---|---|
| Shellfish Species | Diploid | Triploid | Enhancement | Diploid | Triploid | Enhancement |
| Philippines hooded oysters | 40.3 | 64.08 | 59% | 1.12 | 21.36 | 1900% |
| Mediterranean mussels | 89.2 | 138.26 | 55% | 2.97 | 27.65 | 900% |
| Quahog clams | 73.8 | 109.22 | 48% | 2.38 | 18.2 | 800% |
| Queen scallops | 89 | 140.62 | 58% | 2.47 | 23.44 | 900% |

### Example 6

Triploids of Ostrea edulis are produced following essentially the same procedure as described in Example 2 wherein diploids are mated with tetraploids.

### Example 7

Triploids of Ostrea iridescens are produced following essentially the same procedure as described in Example 2 wherein diploids are mated with tetraploids.

### Example 8

Triploids of Ostrea palmula are produced following essentially the same procedure as described in Example 2 wherein diploids are mated with tetraploids.

### Example 9

Triploids of Ostrea folium are produced following essentially the same procedure as described in Example 2 wherein diploids are mated with tetraploids.

### Example 10

Triploids of Mangrove oyster Crassostrea rhizophorae are produced following essentially the same procedure as described in Example 2 wherein diploids are mated with tetraploids.

### Example 11

Triploids of Mangrove oyster Crassostrea brasiliana are produced following essentially the same procedure as described in Example 2 wherein diploids are mated with tetraploids.

### Example 12

Triploids of Suminoe oyster Crassostrea ariakensis are produced following essentially the same procedure as described in Example 2 wherein diploids are mated with tetraploids.

### Example 13

Triploids of Saccostrea palmipes are produced following essentially the same procedure as described in Example 2 wherein diploids are mated with tetraploids.

### Example 14

Triploids of Saccostrea echinata are produced following essentially the same procedure as described in Example 2 wherein diploids are mated with tetraploids.

### Example 15

Triploids of Blood cockle Anadara granosa are produced following essentially the same procedure as described in Example 2 wherein diploids are mated with tetraploids.

### Example 16

Triploids of Manila clam Venerupis philippinarum are produced following essentially the same procedure as described in Example 2 wherein diploids are mated with tetraploids.

### Example 17

Triploids of Zebra mussel Dreissena polymorpha are produced following essentially the same procedure as described in Example 2 wherein diploids are mated with tetraploids.

### Example 18

Triploids of Quagga mussel Dreissena bugensis are produced following essentially the same procedure as described in Example 2 wherein diploids are mated with tetraploids.

### Example 19

Triploids of Blue mussel Mytilus edulis are produced following essentially the same procedure as described in Example 2 wherein diploids are mated with tetraploids.

### Example 20

Triploids of Korean mussel Mytilus coruscus are produced following essentially the same procedure as described in Example 2 wherein diploids are mated with tetraploids.

### Example 21

Triploids of Asian green mussel Perna viridis are produced following essentially the same procedure as described in Example 2 wherein diploids are mated with tetraploids.

### Example 22

Triploids of Chilean mussel Mytilus chilensis are produced following essentially the same procedure as described in Example 2 wherein diploids are mated with tetraploids.

### Example 23

Triploids of Bay scallop Argopecten irradians are produced following essentially the same procedure as described in Example 2 wherein diploids are mated with tetraploids.

### Example 24

Triploids of Peruvian scallop Argopecten purpuratus are produced following essentially the same procedure as described in Example 2 wherein diploids are mated with tetraploids.

EXAMPLE 25

Triploids of Japanese scallop Patinopecten yessoensis are produced following essentially the same procedure as described in Example 2 wherein diploids are mated with tetraploids.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for producing a triploid oyster, the method comprising mating a tetraploid oyster with a diploid oyster, wherein the triploid oyster belongs to a genus from the group consisting of Ostrea, Crassostrea, and Saccostrea.

2. The method of claim 1, wherein the triploid oyster belongs to a species from the group consisting of Ostrea edulis, Ostrea iridescens, Ostrea palmula, and Ostrea folium.

3. The method of claim 1, wherein the triploid oyster belongs to a species from the group consisting of Crassostrea rhizophorae, Crassostrea brasiliana, and Crassostrea ariakensis.

4. The method of claim 1, wherein the triploid oyster belongs to a species from the group consisting of Saccostrea malabonensis, Saccostrea palmipes, and Saccostrea echinate.

5. The method of claim 1, wherein the triploid oyster belongs to the species Saccostrea malabonensis.

6. A method for producing a triploid shellfish, the method comprising mating a tetraploid shellfish with a diploid shellfish, wherein the triploid shellfish belongs to a genus from the group consisting of Anadara, Venerupis, Mercenaria, Dreissena, Mytilus, and Perna.

7. The method of claim 6, wherein the triploid shellfish belongs to the species Anadara granosa.

8. The method of claim 6, wherein the triploid shellfish belongs to the species Venerupis philippinarum.

9. The method of claim 6, wherein the triploid shellfish belongs to the species Mercenaria mercenaria.

10. The method of claim 6, wherein the triploid shellfish belongs to a species from the group consisting of Dreissena polymorpha and Dreissena bugensis.

11. The method of claim 6, wherein the triploid shellfish belongs to a species from the group consisting of Mytilus edulis, Mytilus galloprovincialis, Mytilus chilensis, and Mytilus coruscus.

12. The method of claim 6, wherein the triploid shellfish belongs to the species Mytilus galloprovincialis.

13. The method of claim 6, wherein the triploid shellfish belongs to a species from the group consisting of Perna viridis and Perna canaliculus.

14. A method for producing a triploid scallop, the method comprising mating a tetraploid scallop with a diploid scallop, wherein the triploid scallop belongs to the species from the group consisting of Argopecten irradians, Argopecten opercularis, Argopecten purpuratus, and Patinopecten yessoensis.

15. The method of claim 14, wherein the triploid scallop belongs to the species Argopecten irradians.

16. The method of claim 14, wherein the triploid scallop belongs to the species Argopecten opercularis.

17. The method of claim 14, wherein the triploid scallop belongs to the species Argopecten purpuratus.

18. The method of claim 14, wherein the triploid scallop belongs to the species Patinopecten yessoensis.
